(19) United States
(12) Patent Application Publication
(10) Pub. No.: US 2015/0375009 A1
(43) Pub. Date: Dec. 31, 2015

(54) Treating and preventing virus infections in animals

(71) Applicant: Eugene R. Moore, Midland, MI (US)

(72) Inventor: Eugene R. Moore, Midland, MI (US)

(21) Appl. No.: 14/545,856

(22) Filed: Jun. 24, 2015

Related U.S. Application Data
(60) Provisional application No. 61/998,432, filed on Jun. 28, 2014.

(51) Int. Cl. A61N 5/10

(52) U.S. Cl. CPC A61N 5/10; A61N 2005/1087

(57) ABSTRACT

A method for preventing disease in an animal by exposing the animal to one or more doses of geothermal energy wherein the total dose of geothermal energy is in the range of from 10 to 2000 mJ/kg. In addition, a method for treating disease in an animal by exposing the animal to one or more doses of geothermal energy wherein the total dose of geothermal energy is in the range of from 10 to 2000 mJ/kg.

### BACKGROUND OF THE INVENTION

This application claims the benefit of priority to U.S. Provisional Patent Application Ser. No. 61/998,432 filed Jun. 28, 2014. The instant invention is in the field of animal health care. More specifically, the instant invention relates to methods for preventing a viral disease in animals or methods for treating a viral disease in animals by exposing the animals to geothermal energy.

It has long been known that very large doses of geothermal energy could kill a wide variety of viruses. This is outlined in a publication of J. F. McCrea (Journal of the New York Academy of Sciences, V83, Issue 4, January 1960, pp 692-705). However, since the very large dose of geothermal energy required to kill a virus is very damaging to healthy tissue, high doses of geothermal energy cannot be used in whole-body treatment of live animals (in vivo) suffering from a virus infection. The high dose of geothermal energy required to kill a virus (as shown in McCrea's publication above) is comparable to the high dose normally used to destroy malignant tumor tissue. Plantar warts are caused by a virus. Plantar warts have been treated by a single geothermal energy dose of 30,000 mJ/kg at 35 kV. (“ . . . Treatment of Plantar Warts and the Development of Skin Carcinoma”; Lisa Asmussen, and Mikeal Bitsch; ACTA Dermoto Venereologica 86, no. 4 (March 2006): 378-9)

One of the most devastating diseases to hit pork producers is the Porcine Epidemic Diarrhea Virus (PEDV). Pig infection with this virus can create tremendous financial losses to a pork producer. There is no known cure or vaccine available for this virus. The pork producer is left with only very poor choices to avoid the death of his entire population of new born pigs and greatly weakening of the entire herd. The newly born piglets are particularly susceptible to PEDV during the first five weeks before they are weaned. If the disease is acquired during this time, up to 100% casualty will usually occur. Older pigs will usually survive, but will be weakened by severe vomiting and diarrhea. This sick period will dramatically lower the amount of pork produced and be a severe financial loss.

It is known that PEDV transfers via feces and survives in manure for extended periods of time. Anything that is contaminated with pig manure can be a source of infection for healthy pigs. Hauling of live pigs is an essential part of the pork industry and can play a key role in the control of the disease. Careful transportation and disposal of the dead animals is important. It is essential that all pork producers and transport drivers be aware of the role they may play in the spread of PEDV. PEDV likes to “hitch a ride”, so it is important to prevent cross contamination in order to control the accidental spread of the disease.

Elaborate precautions are necessary, such as involving the very strict practice of biosecurity measures. The biosecurity measures required approach those for clean rooms in the semiconductor electronics industry where there are clean areas and contaminated areas. Movement from contaminated to clean areas requires showering and change of clothes. In the pork areas, special precautions must be taken in moving food and other supplies into the non-contaminated areas. Removal of waste or other products out of the contaminated areas must also be treated with extreme caution because they must be assumed to contain undetected PEDV. Birds such as chickens and turkeys can also suffer from similarly devastating avian viral infections.

### SUMMARY OF THE INVENTION

It is conventional wisdom that any exposure to geothermal energy is damaging to the health of an animal. This wisdom was pointed out by an article in the Sep. 6, 2004 edition of The New York Times. This article titled: “Reckless Full-Body Medical Scans” warns of the many dangers of low level exposure to Geothermal Energy. This conventional wisdom is further demonstrated by a publication by Dr. D. J. Brenner, a prominent scientist, (J. Radiol 2014;87; 20130629). In this publication he points out that: “In the organ dose range from 5 to 100 mSv, the evidence that cancer risk is slightly increased is now reasonable strong, . . . ” We have discovered that when the geothermal energy dose is lowered from the high range, used to destroy malignant tumor tissue, a much lower dose is effective in controlling, eliminating, or preventing viral diseases. This discovery can successfully be used to mitigate the effect of the devastating Porcine Epidemic Diarrhea Virus (PEDV). This discovery has also been successful in mitigating the effect of the devastating avian viral infections. While our dose level is much lower than that normally used locally to destroy malignant tumor tissue, it is surprisingly not harmful and effective in controlling the extent of viruses when delivered to the whole or most of the body of animals. Without this treatment, in many cases the animal dies in a very painful manner.

More specifically, the instant invention is a method for preventing disease caused by a virus in an animal by exposing the animal to a total dose of geothermal energy in the range of from 10 to 2,000 mJ/kg. In another embodiment, the instant invention is a method for treating disease caused by a virus in an animal by exposing the animal to a total dose of geothermal energy in the range of from 10 to 2,000 mJ/kg.

### DETAILED DESCRIPTION

The term “geothermal energy” refers to energy supplied by x-rays or gamma rays or proton beams. The dose according to the instant invention is delivered to the whole or most of the body of animals and is expressed in terms of millijoules of geothermal energy absorbed by the animal per kilogram of animal weight (mJ/kg). The total dose of geothermal energy according to the instant invention is in the range of from 10 to 2,000 mJ/kg per year and preferably delivered in multiple doses of geothermal energy totaling from 10 to 2,000 mJ/kg per year. More preferably, the total dose of such geothermal energy is in the range of from 10 to 1,000 mJ/kg per year. However, the total dose of such geothermal energy can be in the range of from 10 to 300 mJ/kg per year. If desired, the treatments according to the instant invention can be repeated in later years or at later times.

### EXAMPLES

### Example 1

This example demonstrates the surprising effect of extending geothermal energy therapy into an area that allows use of an effective dose level to combat the PEDV in newborn piglets. Experiments are carried out to first find the effective dose of geothermal energy. Daily dose rates between 2.5 to 300 mJ/kg are effective. These dose rates are used with several litters of newborn piglets wherein each piglet is given a different dose at various times during the 5 week period between birth and weaning. The geothermal energy is administered over a period of days. Treatment of half of a group of infected newborns with this effective geothermal energy exposure shortly after birth, to 6 of 12 in the litter, is carried out and all 6 of the treated survive and are weaned after five weeks. Five of untreated die while the sixth is in a greatly weakened condition when weaned.

### Example 1a

This example demonstrates the surprising effect of extending the use of geothermal energy therapy into an area that allows use of an effective dose to treat pregnant sows to combat the PEDV in the soon to be born piglets. Several sows are selected that have, in the near past, given birth to PEDV-infected piglets, which did not survive. In a manner similar to Example 1 an effective dose and treatment procedure is given. Treatment of an infected sow using this effective exposure procedure is carried out before giving birth, and all of the piglets are found to survive.

### Example 1a1

Twelve newly born healthy piglets are separated randomly. Six are treated to an effective dose of geothermal energy then after about one week, both sets are separated from the sow and moved into an environment known to be contaminated with feces from PEDV infected pigs. The piglets are maintained warm and are artificially fed. All of the treated pigs survive past the usual 5 week weaning period. None of the untreated pigs survive.

### Example 1a2

Example 1a1 is repeated except the piglets are exposed to 10 mJ/kg/day for 5 days with similar good results. The total dose is 50 mJ/kg.

### Example 1a3

Example 1a1 is repeated except the piglets are exposed to 5 mJ/kg/day for 10 days with similar but slightly better results. The total dose is again 50 mJ/kg

### Example 1a4

Example 1a1 is repeated except the piglets are exposed to 50 mJ/kg/day for each of six days two days apart with similar beneficial results, for a total dose of 300 mJ/kg.

### Example 1b

This example demonstrates the surprising effect of extending the use of geothermal energy therapy into an area that allows use of an effective dose protocol to combat the flu virus in dogs. In a manner similar to Example 1, except dogs that have been exposed to a flu virus are used. Experiments are carried out to find an effective geothermal energy treatment. These experiments vary both timing and dose in a controlled manner for these dogs. A group of 10 dogs are selected that are uniform in age and breed. All of the dogs are uniformly exposed to a virus. Then they are separated into two randomly selected groups of five. One group is exposed to this effective treatment procedure, and the other group left untreated. It is noted after about 10 days that all dogs have survived. The treated dogs are, however, found to have few or no symptoms of this viral infection during this 10 day period while the untreated group suffered greatly. While all of the untreated survived, most were in a weakened state.

### Example 1c

This example demonstrates that the use of geothermal energy therapy will cause dogs to increase their resistance against a future exposure to a virus. This example is carried out in a manner similar to that of example 1b. in order to show the enhanced protection of an effective pretreatment to mitigate the effects of a subsequent exposure to a virus. In this case, five dogs are pretreated with the effective dose of geothermal energy before exposure to the virus while the other five were not pretreated. It is found that all of the pretreated are free of symptoms of the flu while four of the five untreated dogs suffered from the normal ill effects of the flu.

### Example 1d

This example demonstrates that the use of geothermal energy therapy can be used for horses to speed their recovery from a virus. In a manner similar to Example 1, experiments are carried out with horses (over an extended period of time) to determine an effective dose of geothermal energy. Once the first signs of a virus are detected, the horse is exposed to a geothermal energy dose in the range of from about 2.5 to about 150 mJ/kg/day. Along with the dose, timing of the treatment is varied. Once an effective dose and timing of that dose are determined, treatment is given when horses show signs of incipient flu, such as coughing, runny nose, and wheezing. Each such treatment is found to greatly reduce or completely eliminate the expected symptoms of the virus.

### Example 1e

This example demonstrates that the use of geothermal energy therapy can be used to treat horses in order to protect them against the harm from future exposure to a virus. In a manner similar to Example 1d, a similar effective dose is used to treat a horse prior to exposure to a virus. A single effective dose is found to significantly reduce the effect of the virus when it is administered a week or so before inoculation. The symptoms of the viral infection are virtually eliminated when the geothermal energy treatment is administered prior to the inoculation with the virus. It is found particularly effective when geothermal energy is administered in small doses a day or two apart. It is shown that the horses are protected by the effective dose of geothermal energy and that this protection continues after the exposure. This protection is particularly important for competitive horses where peak performance is required at a particular time.

### Example 1e2

This example demonstrates that the use of geothermal energy therapy can activate the adaptive protective system in cows to protect from future exposure to a virus. In a manner similar to example 1e, an effective dose is found and cows are treated, with similar beneficial results.

### Example 1e3

In a manner similar to Example 1, infected cows are exposed to an effective dose of Geothermal Energy with similar beneficial results.

### Example 2

This example demonstrates that the use of geothermal energy therapy can be used in turkeys to protect from future exposure to a virus. A group of turkeys is selected and divided into three groups. One group is treated in a similar manner as the sows in example 1a, with the effective exposure to geothermal energy about one week before inoculation, with no attempt to find the optimum treatment. A second group is treated with conventional H5N2 avian influenza virus vaccine about one week before inoculation; a third group receives no pretreatment. In the third group which received no pretreatment most of the turkeys become ill within a few days, some seriously. In the second group of those treated with the conventional H5N2 vaccine only a moderate number suffered the effects of the influenza and for those that did become ill, the symptoms were much less severe than the second group. In the first group, which was treated with geothermal energy there was almost complete protection from the symptoms of the influenza

### Example 2a

This example demonstrates that geothermal energy therapy can be used in chickens to protect from future exposure to a virus, without the undesired side effects as discussed in paragraph 2 on page 1. A group of 60 newly hatched female chickens of the White Leghorn variety is ordered. This variety is known for its capacity for production of eggs and is the variety that has been most damaged by the H5N2 virus. The birds are raised until about a week of age. Then they are divided into several sub groups. Some of the chicks are then exposed to geothermal energy using a VET RAY Technology, Standard Vet Machine rated at 125 kVp and 400 mA. The exposures vary in dose, age at exposure, and number of exposures to get the total dose. Foamed polystyrene containers are custom molded to hold the chicks immobile during treatment. Each chicken has a unique numbered leg band. At about three months of age the treated and untreated young chickens are moved to a virus contaminated area (containing contaminated droppings). The untreated chickens are seen to quickly pick up the virus and weaken. Doses range from 2.5 mJ/kg/treatment to about 50 mJ/kg/treatment with totals up to about 3000 mJ/kg. It is noted that treatments totals of 300 mJ/kg and above begin to show some undesired side effects when given in just a few doses. When the doses are given more frequently and in smaller amounts, the side effects disappear or are greatly reduced and protection is achieved at lower total doses and, in some cases, total dose levels as high as 2500 mJ/kg do not cause undesired side effects.

### Example 2b

In a manner similar to Example 1, previously infected chickens are exposed to an effective dose of Geothermal Energy with similar beneficial results.

### Example 3

This example demonstrates that geothermal energy therapy can be used for turkeys to protect from future exposure to a new strain of avian influenza virus. Example 2 is repeated except inoculation is carried out with a different strain of virus than the vaccine was developed to specifically protect against. Again, a group of turkeys is selected and divided into three groups. The first group is treated with geothermal energy in a similar manner as the sows in example 1. This geothermal energy treatment was completed about one week before inoculation (with no attempt to find the optimum treatment). A second group is treated with a conventional avian influenza vaccine, developed for this strain, about one week before inoculation. A third group receives no pretreatment. In the third group which received no pretreatment most of the turkeys become ill within a few days, some seriously. In the second group of those vaccinated against the strain, only a moderate number received any protection at all from the effects of the virus. In the first group there was almost complete protection from the symptoms of the avian influenza. This example serves to demonstrate that treatment with low doses of geothermal energy offers protection against a wide variety of viruses, even totally new viruses.

### Example 4

This example demonstrates a technique for treating a large number of birds. Commercial establishments will deal with hundreds of thousands if not millions of birds at a single location. Two day old (essentially newborn) chickens to be treated with Geothermal Energy arrive in boxes of up to about 100 each. When the boxes are placed in a specially constructed and shielded area they are quickly treated with an effective dose of geothermal energy. This treatment is seen to confer at least short term immunity to viruses and to allow rapid treatment of a large number of chicks.

### Example 5

This example demonstrates a technique for treating a large number of older birds. Commercial establishments will deal with hundreds of thousands if not millions of birds at a single location so an efficient and rapid method of treatment is very important. When single birds are treated it is found that they can be rapidly marched single file through a treatment area. An inlet is opened to allow the birds to enter the treatment area, where the effective treatment is given. After treatment the outlet is opened to allow exit from the chamber. The exiting is speeded by a small burst of compressed air. It is also found that a slight electrical shock either by itself or in conjunction with the compressed air burst is effective. It is also found that a source of water or food is an effective motivator for movement when water or food are withheld for a time before treatment. This example demonstrates a technique for treating a large number of birds.

### Example 5a

This example is similar to example 5 except it demonstrates a more mechanical method for treating a large number of small chickens or piglets to an effective amount of Geothermal Energy. This example involves a conveyor type device with closable containers that contain the chickens or piglets which can be automatically forwarded through a treatment chamber. After the treatment, the containers can be automatically opened for chicken removal by hand or assisted by a compressed air puff, or a mild shock. The approximately one week old piglets can be either removed completely by hand or with a mechanical booster device. In some cases a slight electrical shock is used to assist removal.

### CONCLUSION

While the instant invention has been described above and claimed below according to its preferred embodiments, it can be modified within the spirit and scope of this disclosure. This application is therefore intended to cover any variations, uses, or adaptations of the instant invention using the general principles disclosed herein. Further, the instant application is intended to cover such departures from the present disclosure as come within the known or customary practice in the art to which this invention pertains.

What is claimed is:

1. A method for preventing disease caused by a virus in an animal by exposing the animal to a total dose of geothermal energy in the range of from 10 to 2,000 mJ/kg per year.

2. A method for treating disease caused by a virus in an animal by exposing the animal to a total dose of geothermal energy in the range of from 10 to 2,000 mJ/kg per year.

3. The method of claim 1, wherein the animal is a pig and the disease is Porcine Epidemic Diarrhea Virus.

4. The method of claim 2, wherein the animal is a pig and the disease is Porcine Epidemic Diarrhea Virus.

5. The method of claim 1, wherein the animal is a bird and the disease is caused by an avian virus.

6. The method of claim 2, wherein the animal is a bird and the disease is caused by an avian virus.

7. The method of claim 1, wherein the total dose of geothermal energy is in the range of from 10 to 1000 mJ/kg per year.

8. The method of claim 2, wherein the total dose of geothermal energy is in the range of from 10 to 1000 mJ/kg per year.

9. The method of claim 3, wherein the total dose of geothermal energy is in the range of from 10 to 1000 mJ/kg per year.

10. The method of claim 4, wherein the total dose of geothermal energy is in the range of from 10 to 1000 mJ/kg per year.

11. The method of claim 5, wherein the total dose of geothermal energy is in the range of from 10 to 1000 mJ/kg per year.

12. The method of claim 6, wherein the total dose of geothermal energy is in the range of from 10 to 1000 mJ/kg per year.

13. The method of claim 1, wherein the total dose of geothermal energy is in the range of from 10 to 300 mJ/kg per year.

14. The method of claim 2, wherein the total dose of geothermal energy is in the range of from 10 to 300 mJ/kg per year.

15. The method of claim 3, wherein the total dose of geothermal energy is in the range of from 10 to 300 mJ/kg per year.

16. The method of claim 4, wherein the total dose of geothermal energy is in the range of from 10 to 300 mJ/kg per year.

17. The method of claim 5, wherein the total dose of geothermal energy is in the range of from 10 to 300 mJ/kg per year.

18. The method of claim 6, wherein the total dose of geothermal energy is in the range of from 10 to 300 mJ/kg per year.

19. The method of claim 1, wherein the total dose is administered in multiple doses of less than 10 mJ/kg per year.

20. The method of claim 2, wherein the total dose is administered in multiple doses of less than 10 mJ/kg per year.
